(19) United States
(12) Patent Application Publication
(10) Pub. No.: US 2014/0147508 A1
(43) Pub. Date: May 29, 2014

(54) Nano-Carrier, Complex of Anticancer Drug and Nano-Carrier, Pharmaceutical Composition Thereof, Method for Manufacturing the Complex, and Method for Treating Cancer by Using the Pharmaceutical Composition

(71) Applicant: National Cheng Kung University, Tainan (TW)

(72) Inventors: Dar-Bin SHIEH, Tainan (TW); Chen-Sheng YEH, Tainan (TW); Dong-Hwang CHEN, Tainan (TW); Ya-Na WU, Tainan (TW); Ping-Ching WU, Kaohsiung (TW)

(73) Assignee: National Cheng Kung University, Tainan (TW)

(21) Appl. No.: 14/167,116

(22) Filed: Jan. 29, 2014

Related U.S. Application Data
(63) Continuation-in-part of application No. 12/805,424, filed on Jul. 30, 2010, now Pat. No. 8,673,358.

(51) Int. Cl. A61K 47/48; A61K 9/51; A61K 31/513

(52) U.S. Cl. CPC A61K 47/4823; A61K 31/513; A61K 9/51
USPC 424/493; 514/44 R; 536/23.1

(57) ABSTRACT

The present invention relates to a nano-carrier for an anticancer drug, which comprises: a metal nanoparticle; and a polynucleotide for connecting with an anticancer drug having a pyrimidine group or a purine group, wherein the polynucleotide is connected to a surface of the metal nanoparticle, and the anticancer drug is bound to the polynucleotide through the pyrimidine group or the purine group. In addition, the present invention also provides a complex of an anticancer drug and a nano-carrier, a pharmaceutical composition thereof, a method for manufacturing the complex, and a method for treating a cancer by using the pharmaceutical composition.

This application is a continuation-in-part application, and claims benefit of U.S. patent application Ser. No. 12/805,424, filed Jul. 30, 2010, entitled “NANO-CARRIER, COMPLEX OF ANTICANCER DRUG AND NANO-CARRIER, PHARMACEUTICAL COMPOSITION THEREOF, METHOD FOR MANUFACTURING THE COMPLEX, AND METHOD FOR TREATING CANCER BY USING THE PHARMACEUTICAL COMPOSITION,” by Dar-Bin SHIEH, Chen-Sheng YEH, Dong-Hwang CHEN, Ya-Na WU, and Ping-Ching WU, the status of which is pending, the disclosure of which is hereby incorporated herein in its entirety by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### BACKGROUND OF THE INVENTION

1. Field of the Invention

The present invention relates to a nano-carrier, a complex of an anticancer drug and a nano-carrier, a pharmaceutical composition thereof, a method for manufacturing the complex, and a method for treating a cancer by using the pharmaceutical composition and, more particularly, to a nano-carrier which is able to carry an anticancer drug and release the anticancer drug near the location of cancer cells, a complex of an anticancer drug and a nano-carrier, a pharmaceutical composition thereof, a method for manufacturing the complex, and a method for treating a cancer by using the pharmaceutical composition

2. Description of Related Art

Chemotherapeutic agents are cytotoxic drugs that usually target fast growing cells through blockage of critical pathways for cell division as well as promoting apoptosis. One kind of the widely used chemotherapeutic agents serving as anti-virus and/or anti-cancer drugs is nucleotide-like compounds. The nucleotide-like compounds can kill the virus-infected cells and/or cancer cells or inhibit the growth thereof through interfering with the nucleic acid metabolisms and cell divisions, and promoting apoptosis. However, the nucleotide-like compounds are also cytotoxic to normal cells, so some side effects and complications may occur during the administration of these compounds, and the dosages thereof must be limited. Also, some nucleotide-like compounds may interfere with the gene replication and transcription in mitochondrions and cell nucleuses when these compounds are administered over a long period of time. Hence, some side effects, such as mitochondrial disorder and bone marrow suppression, may occur in the patients taking these compounds for a long time.

In the clinical researches, it is found that when the nucleotide-like compounds are used in the anti-virus therapy, drug-resistant strains of virus may exist in some patients taking drugs with a single-agent for a long time. Hence, the activity of the virus in serum increases again, the patient's condition gets worse, and the drugs have to be replaced by a new agent. In addition, when the nucleotide-like compounds used as an anti-cancer drug are administered for a long time, cancer cells with drug-resistant strains may be generated and cause the efficacy of the chemical therapy to decrease. Furthermore, the nucleotide-like compounds attack both normal and cancerous cells thereby often resulting in significant side effects, notably lethal cardiac toxicity. Hence, the dose of the anti-cancer drug must be limited to prevent side effects.

Fluorouracil (5-FU) is one kind of nucleotide-like compounds, which is the main ingredient for treating GI tract cancers, including colorectal, stomach, and oral cancers in the past decades. 5-FU is a pyrimidine analogue and can be converted in the cancer cell to form cytotoxicity metabolites, which then become incorporated into DNA and RNA. The compounds eventually induce cell cycle arrest and apoptosis by inhibiting DNA synthesis.

Therefore, it is desirable to provide an anti-cancer drug, and the drug release strategies thereof can be controlled and sustained to increase local concentration of anti-cancer drugs, in order to decrease potential side effects.

### SUMMARY OF THE INVENTION

The object of the present invention is to provide a nano-carrier for an anticancer drug, a complex of an anticancer drug and a nano-carrier, a pharmaceutical composition for treating a cancer, which have the properties of the controlled drug release and can be used to treat cancer cell localizedly.

Another object of the present invention is to provide a method of manufacturing a complex of an anticancer drug and a nano-carrier, which can be used to prepare a complex with the property of controlled drug release.

A further object of the present invention is to provide a method of treating a cancer, which can be used to localizedly treat cancer cells in patients.

To achieve the object, the nano-carrier for an anticancer drug of the present invention comprises: a metal nanoparticle; and a polynucleotide for connecting with an anticancer drug having a pyrimidine group or a purine group, wherein the polynucleotide is connected to a surface of the metal nanoparticle, and the anticancer drug is bound to the polynucleotide through the pyrimidine group or the purine group.

In addition, the complex of an anticancer drug and a nano-carrier of the present invention comprises: a nano-carrier comprising a metal nanoparticle, and a polynucleotide is connected to a surface of the metal nanoparticle; and an anticancer drug with a pyrimidine group or a purine group, wherein the anticancer drug is bound to the polynucleotide through the pyrimidine group or the purine group.

The present invention also provides a pharmaceutical composition for treating a cancer, which comprises the aforementioned complex of an anticancer drug and a nano-carrier.

Further, the method of manufacturing a complex of an anticancer drug and a nano-carrier of the present invention comprises the following steps: (A) providing a nano-carrier, and an anticancer drug, wherein the nano-carrier comprises a metal nanoparticle, and a polynucleotide is connected to a surface of the metal nanoparticle, and the anticancer drug comprises a pyrimidine group or a purine group; and (B) mixing the anticancer drug with the nano-carrier to obtain a complex of the anticancer drug and the nano-carrier, wherein the anticancer drug is binds to the polynucleotide through the pyrimidine group or the purine group.

In addition, the method for treating a cancer of the present invention comprises: providing the aforementioned pharmaceutical composition to a patient.

According to the nano-carrier, the complex of an anticancer drug and the nano-carrier, and a pharmaceutical composition thereof of the present invention, the nano-carrier can carry the anticancer drug and release the anticancer drug near the location of cancer cells. In the present invention, the base group of the polynucleotide is complementary to the pyrimidine group or the purine group anticancer drug, so the nano-carrier of the present invention can carry the anticancer drug. Furthermore, the polynucleotide can serve as a natural biopolymer and decrease the potential toxicity, metabolic clearance and immunological issues. In addition, polynucleotide such as anti-sense oligonucleotide can be applied for the modulation of gene expression. By modification of the polynucleotide sequence used, it is possible to carry different anticancer drugs, and achieve the purposes of integrated gene expressive modification and controlled drug release. Because the anticancer drug can be released localizedly, it is possible to prevent the anticancer drug from attacking the normal cells and reduce the generation of the drug resistance.

According to the present invention, one end of the polynucleotide is connected to the surface of the metal nanoparticle. Herein, the polynucleotide may be connected to the surface of the metal nanoparticle through a covalent bonding, a non-covalent bonding such as hydrogen bonding, or an affinity-adsorption.

According to the present invention, the material of the metal nanoparticle may be any biocompatible material. Preferably, the metal nanoparticle is an Au nanoparticle, Ag nanoparticle, Fe₂O₃ nanoparticle, or a nanoparticle (for example, an Fe core) coated with an Au shell or Ag shell. More preferably, the metal nanoparticle is an Au nanoparticle, Ag nanoparticle, or an Fe₂O₃ nanoparticle. Most preferably, the metal nanoparticle is an Au nanoparticle. In addition, the shape of the metal nanoparticle is not particularly limited. Preferably, the metal nanoparticle is spherical.

Furthermore, according to the present invention, the metal nanoparticle is nano-sized. Preferably, the diameter of the metal nanoparticle is 1-100 nm. More preferably, the diameter of the metal nanoparticle is 1-50 nm. Most preferably, the diameter of the metal nanoparticle is 1-30 nm.

According to the present invention, the sequence of the polynucleotide may be designed according to the anticancer drug. Preferably, the polynucleotide comprises at least one nucleotide selected from the group consisting of adenine and guanine. When the polynucleotide comprises adenines, an anticancer drug named Fluorouracil (5-FU) can bond to the polynucleotides through the pyrimidine group of 5-FU. Therefore, the complex of the 5-FU and the nano-carrier can be used to treat GI tract cancer, and is especially used in chemotherapy for colorectal cancers. When the polynucleotide comprises guanines, cytosine analog can bond to the polynucleotides through the imidazole rings of the guanines.

### BRIEF DESCRIPTION OF THE DRAWINGS

FIG. 1 is a perspective view of a nano-carrier for an anticancer drug of the present invention;

FIG. 2 is a diagram showing the drug loading efficiency of Au nanoparticles and nano-carriers for an anticancer drug of the present invention;

FIG. 3 is a diagram showing the drug releasing rate of 5-FU from nano-carriers of the present invention in a PBS solution with different pH; and

FIG. 4 is a diagram showing the results of the MTT assay of 5-FU and complexes of 5-FU and a nano-carrier of the present invention.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### Embodiment 1

Preparation of Au nanoparticles

Au nanoparticles are reduced from Au salts and prepared through a conventional chemical co-precipitation process. The brief process for preparing Au nanoparticles is illustrated as follows.

50 mL of 38.8 mM trisodium citrate (Sigma Aldrich Inc., USA) solution was added to a boiling HAuCl₄ solution (1 mM, 500 mL), and the color of the resulted solution is yellow. When the original yellow color turned into a burgundy-wine red, the solution was slowly cooled to room temperature, and Au nanoparticles coated with citrate were obtained.

Then, a UV-vis spectrophotometer (NanoDrop™ 1000, NanoDrop Technologies, LLC, USA) was used to measure the absorption spectra of the obtained Au nanoparticles. TEM and photon correlation spectroscopy (Dclsa™ Nano Zeta Potential and Submicron Particle Size Analyzer, Beckman Coulter, Inc. USA) was applied to measure their conformation and distributions of the particle size and the hydrodynamic size.

The absorption spectrum shows that the obtained Au nanoparticles have a characteristic optical absorption peak at 520 nm. In addition, the results of the TEM and the photon correlation spectrum show that the obtained Au nanoparticles have diameters of 12 nm. The mean hydrodynamic size of the Au particles is 25 nm. The Zeta potential (surface charge) of the Au particles is at −9.58±1.68 mV.

### Preparation of a Nano-Carrier for an Anticancer Drug

The poly-A polynucleotide (30 base pairs) with alkane thiol modified 5′ termini (MDBio Inc, Taipei, Taiwan) was dissolved in ddH₂O. For the conjugation of the poly-A polynucleotide to the Au nanoparticles, 20 μL of Au colloids (60 nM) was incubated with 34 μL of poly-A polynucleotides (100 μM) for 24 hrs. The reaction mixture was then added with NaCl solution to a final concentration of 0.05 M, then incubated for 24 hrs at 4° C. The salt concentration was gradually increased to 0.1 M then 2 M for the incorporation of more poly-A polynucleotides on the surfaces of the Au nanoparticles in the following two runs of 8 hrs incubation period. The solution was then centrifuged at 10,000×g for 10 min and the pellet was collected, washed three times with Phosphate Buffered Saline (PBS), and finally dissolved in PBS.

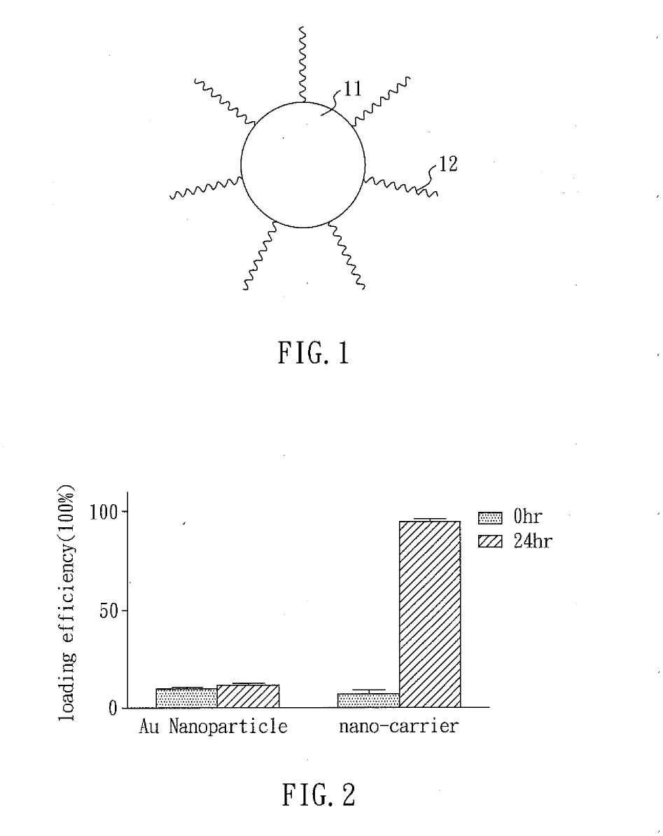

After the aforementioned process, a nano-carrier for an anticancer drug was obtained, which comprises: a metal nanoparticle 11 (Au nanoparticle); and a polynucleotide 12 (poly-A polynucleotides) for connecting with an anticancer drug, wherein the polynucleotide is connected to a surface of the metal nanoparticle, as shown in FIG. 1.

The UV-vis spectrophotometer, TEM and photon correlation spectroscopy are also applied to measure the obtained nano-carrier. The absorption spectrum shows that the nano-carrier has a characteristic optical absorption peak at 525 nm. The mean hydrodynamic size of the nano-carrier is 34 nm. The surface charge of the nano-carrier is −18.19±1.23 mV due to decoration of the negatively charged poly-A polynucleotide.

### Preparation of a Complex of an Anticancer Drug and a Nano-Carrier

A stock solution of Fluorouracil (5-FU) (100 mg/mL) in PBS was provided. The formula of the 5-FU is presented as the following formula (I).

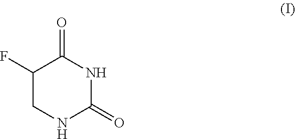

Then, the solution of the 5-FU was added to the aforementioned solution of the nano-carriers (pH 7.0, in PBS) to a final concentration of 10 mg/mL, and then incubated for 24 hrs. Finally, a complex of an anticancer drug (5-FU) and a nano-carrier is obtained, which comprises: a nano-carrier comprising an Au nanoparticle, and poly-A polynucleotides is connected to the surface of the Au nanoparticle; and 5-FU with a pyrimidine group, wherein the 5-FU is bound to the poly-A polynucleotides through the pyrimidine group.

The hydrodynamic size of the complex is 35 nm, and the surface charge is 21.66±2.19 mV. In addition, the absorption spectrum of the complex shows two characteristic optical absorption peaks respectively at 299 nm and 525 nm, wherein 299 nm is the characteristic optical absorption peak of 5-FU, and 525 nm is the characteristic optical absorption peak of the nano-carrier. This result suggests a successful loading of 5-FU onto the nano-carrier.

### Measuring the Anti-Drug Loaded on the Complex

UV-vis spectrophotometric analysis revealed a specific absorption peak of 5-FU at 299 nm and a linear association between OD₂₉₉ and 5-FU concentrations. Au nanoparticles without polynucleotides conjugation served as the control. The amount of 5-FU in the remaining supernatant was measured by spectrophotometer to estimate the amount of drugs loaded onto the nanoparticles. Drug loading (%) efficiency was calculated as: [OD₂₉₉ of the original solution (10 mg/mL 5-FU)-OD₂₉₉ of the supernatant after drug loading]/[OD₂₉₉ of the original solution (10 mg/mL 5-FU)]×100.

The result of the drug loading efficiency is shown in FIG. 2. The results indicated that Au nanoparticles absorbed only 10% of 5-FU in PBS. On the other hand, nano-carrier absorbed about 96% 5-FU after 24 hrs. This result shows that the nano-carrier of the present invention is a high capacity anticancer drug carrier.

Evaluation of the Drug Release from the Nano-Carrier

The drug releasing kinetics of the nano-carrier was evaluated at different pH environment (pH=5, 7, 9) in a PBS buffer in 0.5, 1, 3, 6, 12, 24 and 48 hrs at 37° C. The drug-releasing rate was calculated as: % drug release=[OD₂₉₉ of the supernatant at each time/OD₂₉₉ of the original loaded drugs on the Au-polynucleotide complex]×100. The absorbance of the PBS buffer without drug (OD₂₉₉) was used as a blank, and each absorbance was subtracted with blank.

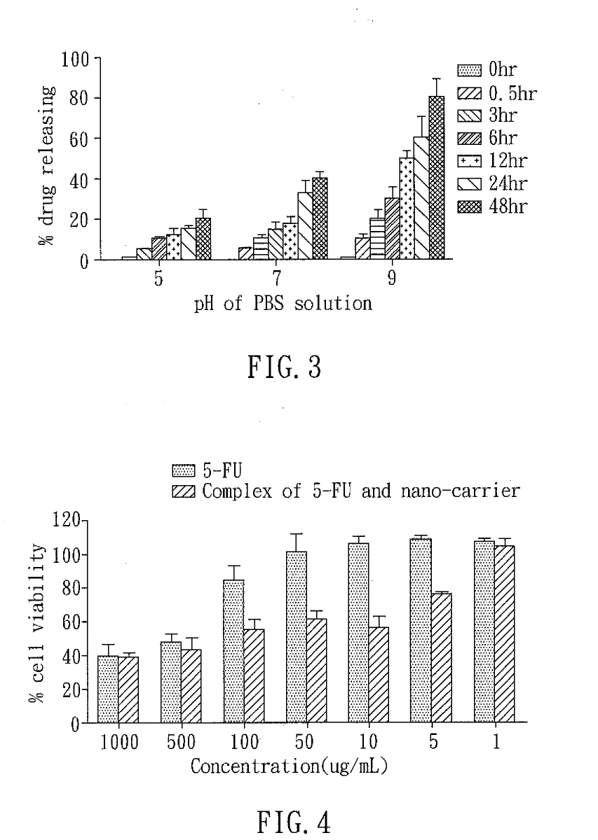

The result of the drug-releasing rate is shown in FIG. 3. According to the results shown in FIG. 3, the loaded 5-FU has a significantly higher release rate in alkaline environment compared to neutral (˜2 folds) and acidic environment (˜4 folds) at 48 hrs. Thus, the nano-carrier of the present invention can serve as an intestine local delivery nano-vehicle to pass through stomach and upper GI tract and then release therapeutic agents (5-FU) in the lower GI tract, thereby being applicable in the per oral chemotherapy for colorectal cancers.

Hence, when the complex of 5-FU and a nano-carrier is applied to treat cancer patients, especially those with colorectal cancers, the 5-FU anticancer drug can be locally released from the complex in lower GI tract and induce cancerous cell cycle arrest.

### In Vitro Cancer Cytotoxicity Analysis of the Complex of 5-FU and the Nano-Carrier

The colon carcinoma cell line SW480 was purchased from the American Type Culture Collection (ATCC). It was maintained in Leibovitz L-15 medium (PAA Laboratories GmbH, Linz, Austria), supplemented with 10% fetal bovine serum (FBS; GIBCO, Taiwan) and 10%, penicillinstreptomycin (100 μg/mL) Cell line was incubated at 37° C. with 5% CO₂ in the air.

The 3-(4,5-dimethylthiazol-2-yl)-2,5-diphenyl tetrazolium bromide (MTT, Sigma Chemical Co., St. Louis, Mo.) assay was performed as described by Mosmann (Mosmann, T. J. Immunol. Methods 1983, 65, 55) with slight modifications (Ulukaya, E.; Colakogullari, M.; Wood, E. J. Chemotherapy (Basel, Switz) 2004, 50, 43). SW480 cancer cells were plated in a 96-well microplate in a final concentration of 5000 cells/well and incubated in a tissue culture incubator overnight. 200 μL medium containing drugs at 4 different concentrations of complexes of 5-FU and nano-carriers were placed in a 96-well plate in triplicate and incubated 24 hrs. The culture medium was replaced by 100 μL of fresh medium. MTT was first prepared as a stock solution of 5 mg/mL in PBS. 20 μL of MTT solution was then added to each well. After incubation for an additional 4 hrs at 37° C., 100 μL of SDS solution (10% sodium dodecyl sulfate dissolved in 0.01 N HCl) was added to each well. After centrifugation at 3,220×g for 5 min, the supernatant were transferred to a new 96-well ELISA plate. Absorbance at 490 nm was measured (LP 400 Pasteur Diagnostics) and calculated. Drug-free complete medium was used as the control (blank) and was treated in the same way as the drug-containing media. 5-FU free compound of the same concentration as those loaded on the nano-carriers was applied as the control for the evaluation of the effect of nano-carriers.

The result of the MTT assay is shown in FIG. 4. According to the results shown in FIG. 4, the complex of 5-FU and the nano-carrier achieves LD (lethal dose) 50% (100 μg/mL) in much lower dosage than 5-FU alone (500 μg/mL). Even at 5 μg/mL of the complex, the therapeutic efficacy still remained 30% in SW480 cancer cell line. Hence, the complex of 5-FU and the nano-carrier of the present invention achieved significantly improved LD₅₀ when compounded to the free 5-FU compound.

In conclusion, according to the aforementioned results, a positive association between environmental pH and drug release was observed in PBS, which implied the potential use in the controlled localized drug release in the lower GI tract. In addition, the MTT assay revealed greater dose dependent cytotoxicity to colon cancer cell line than free compounds, and this suggests the potential use of the complex of the anticancer drug and the nano-carrier as the environmental controlled anti-cancer nanocapsule, which is especially suitable for per oral colon cancer chemotherapy.

### Embodiment 2

The Au nanoparticles used in the present invention were manufactured through the same method as described in the Embodiment 1. The obtained Au nanoparticles have diameters of 13 nm.

The poly-C polynucleotide (15 base pairs) with alkane thiol modified 5′ termini (MDBio Inc, Taipei, Taiwan) was dissolved in ddH₂O. For the conjugation of the poly-A polynucleotide to the Au nanoparticles, 13.68 nM of Au colloids (13 nm) was incubated with 25 μL of poly-C polynucleotides (100 μM) in 10 mM PBS containing 10 mM K₂HPO₄ and 10 mM KH₂PO₄ for 16 hrs. Then, the mixture was adjusted with 2M of NaCl gradient buffer for 64 hrs to saturate the conjugation of the polynucleotides and the nanoparticles. The mixture was adjusted to the concentration of 0.3 M and stayed for 6 hrs. After the conjugation was finished, the mixture was centrifuged with 22000×g for 30 mins, the supernatant was removed, the precipitants were washed with 0.3 M NaCl for 3 times, and the products (i.e. nano-carrier) were washed with de-ionized water for 3 times and ultracentrifuged to concentrate. The precipitants were diluted with 10 mM of PBS, and the diameters and the surface charges of the nanoparticles before and after conjugation with polynucleotides were measured.

The results show that the diameter of the Au nanoparticle is 18.1 nm, and the Zeta potential is −9.58±1.68 mV. After the conjugation, the diameter of the nano-carrier is 100.97 nm, and the Zeta potential is −18.19+1.57 mV.

### Embodiment 3

For the conjugation of the poly-A polynucleotide (15 base pairs) to the Ag nanoparticles, 36.6 nM of Ag nanoparticles (3 nm) was added into 60 μL, of poly-A polynucleotide with alkane thiol modified 5′ termini (100 μM). The poly-A polynucleotide and the Ag nanoparticles were incubated in 10 mM PBS containing 10 mM K₂HPO₄ and 10 mM KH₂PO₄ for 16 hrs. Then, the mixture was centrifuged with 22000×g for 30 mins, the supernatant was removed, and the products (i.e. nano-carrier) were washed with de-ionized water for 3 times and ultracentrifuged to concentrate. The precipitants were diluted with 10 mM of PBS, and the diameters and the surface charges of the nanoparticles before and after conjugation with polynucleotides were measured.

The results show that the diameter of the Ag nanoparticle is 30.57 nm, and the Zeta potential is −29.98±2.12 mV. After the conjugation, the diameter of the nano-carrier is 221.19 nm, and the Zeta potential is −33.62.±2.63 mV.

### Embodiment 4

The Fe₂O₃ nanoparticles used in the present embodiment have diameters of 6.2 nm, and have solubility in water and dispersity. The method and the application of the Fe₂O₃ nanoparticles are the same as those disclosed in TWI 202070.

For the conjugation of the poly-A polynucleotide (15 base pairs) to the Fe₂O₃ nanoparticles, 2 nM of Fe₂O₃ nanoparticles (6.2 nm) was added into 60 μL of poly-A polynucleotide with alkane thiol modified 5′ termini (100 μM). Then, 7 μL of glutaraldehyde (5.5 M) and 14 μL of NaBH₃CN (5.5 M) were added into the mixture, and the reaction was performed for 16 hrs. Then, the mixture was centrifuged with 22000×g for 30 mins, the supernatant was removed, and the products (i.e. nano-carrier) were washed with de-ionized water for 3 times and ultracentrifuged to concentrate. The precipitants were diluted with 10 mM of PBS, and the diameters and the surface charges of the nanoparticles before and after conjugation with polynucleotides were measured. In the present embodiment, the poly-A polynucleotide bond to Fe₂O₃ nanoparticles through covalent bonds. The results show that the diameter of the Fe₂O₃ nanoparticle is 81.2 nm, and the Zeta potential is 31.05±1.35 mV. After the conjugation, the diameter of the nano-carrier is 14773.1 nm, and the Zeta potential is −6.26±1.28 mV.

### Embodiment 5

The Fe₂O₃ nanoparticles used in the present embodiment are the same as those used in the Embodiment 4.

For the conjugation of the poly-A polynucleotide (15 base pairs) to the Fe₂O₃ nanoparticles, 2 nM of Fe₂O₃ nanoparticles (6.2 nm) was added into 60 μL of poly-A polynucleotide without any modification (100 μM). The poly-A polynucleotide and the Fe₂O₃ nanoparticles were incubated in 10 mM PBS containing 10 mM K₂HPO₄ and 10 mM KH₂PO₄ for 16 hrs. Then, the precipitants were diluted with 10 mM of PBS, and the diameters and the surface charges of the nanoparticles before and after conjugation with polynucleotides were measured. In the present embodiment, the poly-A polynucleotide bond to Fe₂O₃ nanoparticles through non-covalent bonds.

The results show that the diameter of the Fe₂O₃ nanoparticle is 90.63 nm, and the Zeta potential is 29.57±2.32 mV. After the conjugation, the diameter of the nano-carrier is 300.97 nm, and the Zeta potential is 10.26±3.65 mV.

Although the present invention has been explained in relation to its preferred embodiment, it is to be understood that many other possible modifications and variations can be made without departing from the scope of the invention as hereinafter claimed.

What is claimed is:

1. A nano-carrier for an anticancer drug, comprising:
a metal nanoparticle; and
a polynucleotide for connecting with an anticancer drug having a pyrimidine group or a purine group, wherein the polynucleotide is connected to a surface of the metal nanoparticle through a covalent bonding or an affinity-adsorption, and the anticancer drug is bound to the polynucleotide through the pyrimidine group or the purine group, wherein the anticancer drug is Fluororacil (5-FU), which is presented as the following formula (I):

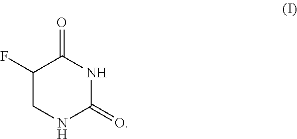

2. The nano-carrier as claimed in claim 1, wherein one end of the polynucleotide is connected to the surface of the metal nanoparticle.

3. The nano-carrier as claimed in claim 1, wherein the metal nanoparticle is an Au nanoparticle, Ag nanoparticle, Fe₂O₃ nanoparticle, or a nanoparticle coated with an Au shell or an Ag shell.

4. The nano-carrier as claimed in claim 1, wherein the diameter of the metal nanoparticle is 1-100 nm.

5. The nano-carrier as claimed in claim 1, wherein the polynucleotide comprises at least one nucleotide selected from the group consisting of adenine and guanine.

6. A complex of an anticancer drug and a nano-carrier, comprising:
a nano-carrier comprising a metal nanoparticle, and a polynucleotide is connected to a surface of the metal nanoparticle through a covalent bonding or an affinity-adsorption; and
an anticancer drug with a pyrimidine group or a purine group, wherein the anticancer drug is bound to the polynucleotide through the pyrimidine group or the purine group wherein the anticancer drug is Fluororacil (5-FU), which is presented as the following formula (I), or Cytosine analog;

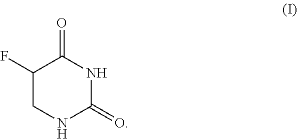

7. The complex as claimed in claim 6 wherein one end of the polynucleotide is connected to the surface of the metal nanoparticle.

8. The complex as claimed in claim 6, wherein the metal nanoparticle is an Au nanoparticle, Ag nanoparticle, Fe₂O₃ nanoparticle, or a nanoparticle coated with an Au shell or an Ag shell.

9. The complex as claimed in claim 6, wherein the diameter of the metal nanoparticle is 1-100 nm.

10. The complex as claimed in claim 6, wherein the polynucleotide comprises at least one nucleotide selected from the group consisting of adenine and guanine.

11. A pharmaceutical composition for treating a cancer, comprising:
a complex of an anticancer drug and a nano-carrier,
wherein the complex comprises: a nano-carrier, and an anticancer drug, the nano-carrier comprises a metal nanoparticle, and a polynucleotide is connected to a surface of the metal nanoparticle through a covalent bonding or an affinity-adsorption, the anticancer drug comprises a pyrimidine group or a purine group, and the anticancer drug is bound to the polynucleotide through the pyrimidine group or the purine group wherein the anticancer drug is Fluororacil (5-FU), which is presented as the following formula (1), or Cytosine analog;

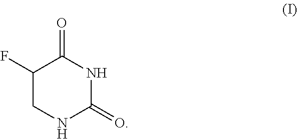

12. The pharmaceutical composition as claimed in claim 11, wherein one end of the polynucleotide is connected to the surface of the metal nanoparticle.

13. The pharmaceutical composition as claimed in claim 11, wherein the metal nanoparticle is an Au nanoparticle, Ag nanoparticle, Fe₂O₃ nanoparticle, or a nanoparticle coated with an Au shell or an Ag shell.

14. The pharmaceutical composition as claimed in claim 11, wherein the diameter of the metal nanoparticle is 1-100 nm.

15. The pharmaceutical composition as claimed in claim 11, wherein the polynucleotide comprises at least one nucleotide selected from the group consisting of adenine and guanine.

16. A method of manufacturing a complex of an anticancer drug and a nano-carrier, comprising the following steps:
(A) providing a nano-carrier, and an anticancer drug, wherein the nano-carrier comprises a metal nanoparticle, and a polynucleotide is connected to a surface of the metal nanoparticle, and the anticancer drug comprises a pyrimidine group or a purine group; and
(B) mixing the anticancer drug with the nano-carrier to obtain a complex of the anticancer drug and the nano-carrier, wherein the anticancer drug is bound to the polynucleotide through the pyrimidine group or the purine group, wherein the anticancer drug is Fluorouracil (5-FU), which is presented as the following formula (I), or Cytosine analog;

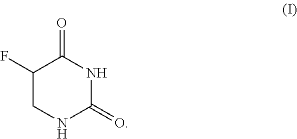

17. The method as claimed in claim 16, wherein one end of the polynucleotide is connected to the surface of the metal nanoparticle.

18. The method as claimed in claim 16, wherein the metal nanoparticle is an Au nanoparticle, Ag nanoparticle, Fe₂O₃ nanoparticle, or a nanoparticle coated with an Au shell or an Ag shell.

19. The method as claimed in claim 16, wherein the diameter of the metal nanoparticle is 1-100 nm.

20. The method as claimed in claim 16, wherein the polynucleotide comprises at least one nucleotide selected from the group consisting of adenine and guanine.

21. A method of treating a cancer, comprising:
providing a pharmaceutical composition comprising: a complex of an anticancer drug, and a nano-carrier, to a patient,
(A) wherein the complex comprises: a nano-carrier, and an anticancer drug, the nano-carrier comprises a metal nanoparticle, and a polynucleotide is connected to a surface of the metal nanoparticle, the anticancer drug comprises a pyrimidine group or a purine group, and the anticancer drug is bound to the polynucleotide through the pyrimidine group or the purine group, wherein the anticancer drug is Fluorouracil (5-FU), which is presented as the following formula (I):

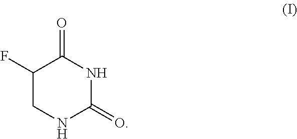

22. The method as claimed in claim 21, wherein the cancer is a GI tract cancer.

23. The method as claimed in claim 21, wherein one end of the polynucleotide is connected to the surface of the metal nanoparticle.

24. The method as claimed in claim 21, wherein the metal nanoparticle is an Au nanoparticle, Ag nanoparticle, Fe₂O₃ nanoparticle, or a nanoparticle coated with an Au shell or an Ag shell.

25. The method as claimed in claim 21, wherein the diameter of the metal nanoparticle is 1-100 nm.

26. The method as claimed in claim 21, wherein the polynucleotide comprises at least one adenine
